(19) United States
(12) Patent Application Publication
(10) Pub. No.: US 2019/0228854 A1
(43) Pub. Date: Jul. 25, 2019

(54) Method For Making A Recommendation For A Package Of Food

(71) Applicant: Worcester Polytechnic Institute, Worcester, MA (US)

(72) Inventors: Prateek Jain, Worcester, MA (US); Soussan Djamasbi, Worcester, MA (US)

(21) Appl. No.: 16/256,805

(22) Filed: Jan. 24, 2019

Related U.S. Application Data
(60) Provisional application No. 62/621,825, filed on Jan. 25, 2018.

(51) Int. Cl. G16H 20/60; G09B 19/00; G16H 50/30; G06Q 30/06

(52) U.S. Cl. CPC G16H 20/60; G09B 19/0092; G06Q 30/0631; G06Q 30/0627; G16H 50/30

(57) ABSTRACT

A recommendation for purchase or selection of a package of food to a consumer, such as a retail of wholesale purchaser of the food package, or a direct or indirect consumer of the food contained in the food package is generated by selecting at least one nutritional value from a food package label by optical character recognition of a representation of the selected nutritional value as it is presented on the package of food. The at least one selected nutritional value is compared to at least one recommended nutritional value corresponding to the selected nutritional value to form at least one comparison value. The comparison value is employed to generate a recommendation that is displayed to the consumer, such as by superimposing the recommendation over an image on a display screen of the food package label as an augmented reality, or in real time, meaning that the recommendation appears simultaneously with the optical character recognition of the nutritional value, wherein the recommendation is displayed with, or is superimposed on an image on the display screen of the representation of the nutritional value, thereby making a recommendation of the food package to the consumer.

### RELATED APPLICATION

This application claims the benefit of U.S. Provisional Application No. 62/621,825 filed Jan. 25, 2018. The entire teachings of the above application are incorporated herein by reference.

### BACKGROUND OF THE INVENTION

Food packaging is subject to government regulation, and labeling requirements often include, as a consequence, a large amount of densely-organized information. One result is that the typical consumer often is unable to quickly and easily discern information that is relevant to personal preferences and needs.

Known methods for sorting information contained on labels of packaged food are often themselves cumbersome and are limited by display of information that is not relevant to the individual consumer trying to make a decision regarding purchase or consumption of a product, such as the types of labels that are suitable for use by the method applied. Further, such methods often cannot be customized to suit individual consumers and, as a result, either omit information that the consumer is seeking to obtain while making a decision to purchase or consume, or presents or incorporates extraneous information that renders the method of limited utility.

Therefore, a need exists for a method of making a purchase or consumption recommendation for a package of food to a consumer that overcomes or minimizes the above-referenced problems.

### SUMMARY OF THE INVENTION

The invention is directed to a method for making a recommendation of a package of food to a consumer.

In one embodiment, the method of the invention includes selecting at least one nutritional value from a food package label of a package of food by optical character recognition of a representation of the selected nutritional value as it is presented on the package of the food. The at least one selected nutritional value is compared to at least one recommended nutritional value corresponding to the selected nutritional value to form at least one comparison value. A recommendation is generated from the at least one comparison value. The recommendation is displayed for the food package to the consumer, wherein the recommendation is displayed with, or is superimposed on an image of the representation of the nutritional value, thereby making a recommendation of the food package to the consumer.

This invention has many advantages. For example, optical character recognition of a representation of a selected nutritional value on a label of a package of food is not dependent upon the appearance of the label itself, such as how the information is organized on the label relative to other nutritional information, nor is it dependent upon the manner in which the information is presented, either as an absolute value, or as a percentage of daily requirements recommended by the Food and Drug Administration (FDA). Further, the information selected from the food package label can be customized according to preferences and needs of the consumer, thereby eliminating generation and presentation of extraneous information from the food label that is not of interest to the consumer when making a decision regarding purchase of a package of food or use of a food contained in the package of food, such as use of the food to prepare a dish or a meal. The recommendation is presented in augmented reality, meaning that the recommendation is displayed with, or is superimposed on an image of the nutritional value simultaneously with the optical character recognition of the nutritional value presented on the package of food. In addition, any overall recommendation made by the method of the invention can be made more useful by a customer who chooses to eliminate from consideration certain nutritional information on the label that is limited in value. Customization by the customer also can include weighting of the nutritional values on the label that are selected for consideration by the consumer and incorporation of those values, as weighted, into a final recommendation. The selected values, and the overall recommendation, can be superimposed on the label without the presence of extraneous information and in real time, thereby greatly facilitating ease of use by the consumer while purchasing packaged food items at a retail site.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic representation of one embodiment of a system of the invention.

FIG. 2A is a perspective view of an individual holding a device in front of a nutrition label of a package of food, wherein representations of selected nutritional values are recognized by optical character recognition and, as a result of the method of the invention, simultaneously displaying, (e.g. in real time), a recommendation on a screen of the device employed to practice the method of the invention.

FIG. 2B is a detail of the perspective view of the representation in FIG. 1A, displaying the result of one embodiment of the invention.

FIG. 3A is a screenshot of another display on a device of a recommendation superimposed in real time over an image of a label on a package of food, wherein the recommendation is presented as a listing of nutritional values that reflect selected representations of nutritional values that appear on the label.

FIG. 3B is a detail of the listing shown in FIG. 2A.

FIG. 4 is partial view of another embodiment of a display generated by a method of the invention showing portions of a nutrition label on a package that have each been framed in color by the method of the invention and assigned color-coded icons (“thumbs-up” or “thumbs-down”), and an overall color-coded recommendation at a top portion of the display signified by an upward-pointing arrow along a graduated scale of color between color-coded icons representing overall negative and positive recommendations.

FIG. 5 is a schematic representation of an algorithm of one embodiment of the method of the invention.

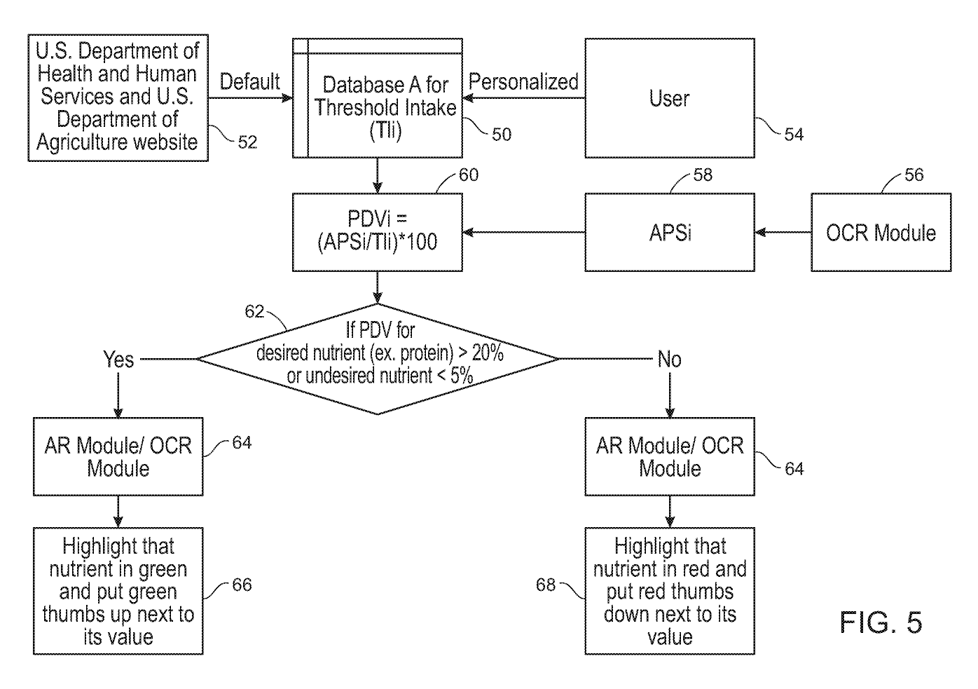

### DETAILED DESCRIPTION

A description of example embodiments follows. While example embodiments have been particularly shown and described, it will be understood by those skilled in the art that various changes in form and details may be made therein without departing from the scope of the embodiments encompassed by the appended claims. The teachings of all patents, published applications and references cited herein are incorporated by reference in their entirety.

The invention generally is directed to a system and method for making a purchase recommendation for a package of food to a consumer, such as a retail or wholesale purchaser of the food package, or a direct or indirect (e.g. the food becomes an ingredient in a dish or meal) consumer of food contained in the labeled package of food.

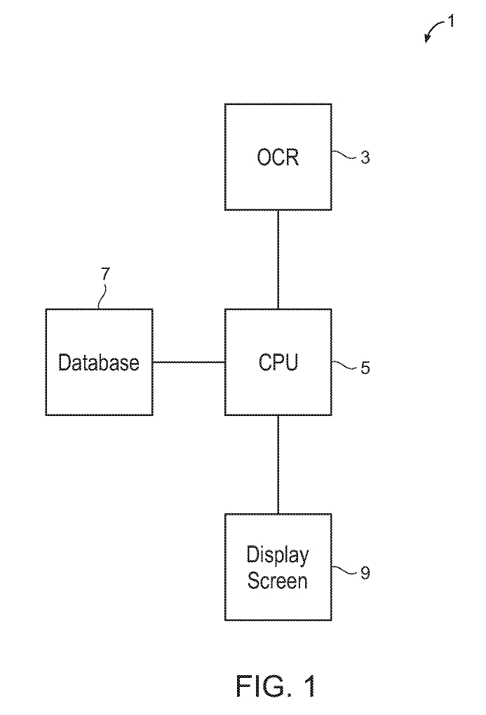

In one embodiment, shown in FIG. 1, system 1 includes optical character recognition device (OCR) 3, central processing unit (CPU) 5, display screen 7, and database 9. CPU 5 links OCR 3, display screen 7 and database 9. CPU 5, OCR 3, display screen 7 and database 9 can all be embodied as a pre-programmed device, such as a handheld device. An example of a suitable handheld device is a “smart phone.”

The manner in which the recommendation is made to the consumer can take various forms, but, in one embodiment of the method, a “smartphone” can be employed to practice the method of the invention by optical character recognition of a representation of selected nutritional values as they are presented on the package of food. Another suitable device for use with the method of the invention are glasses with a heads-up display, also referred to as “smart glasses.”

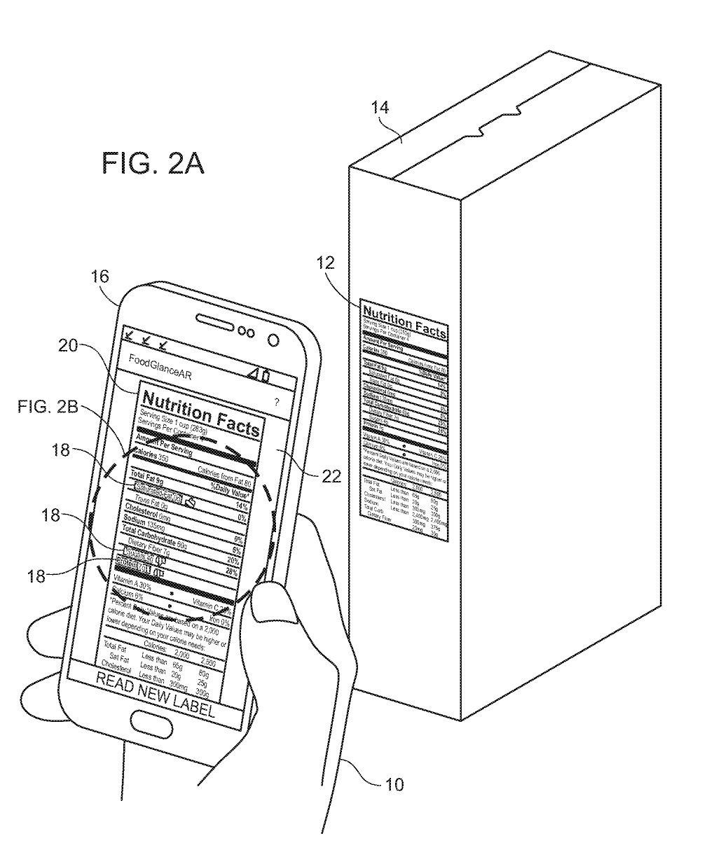

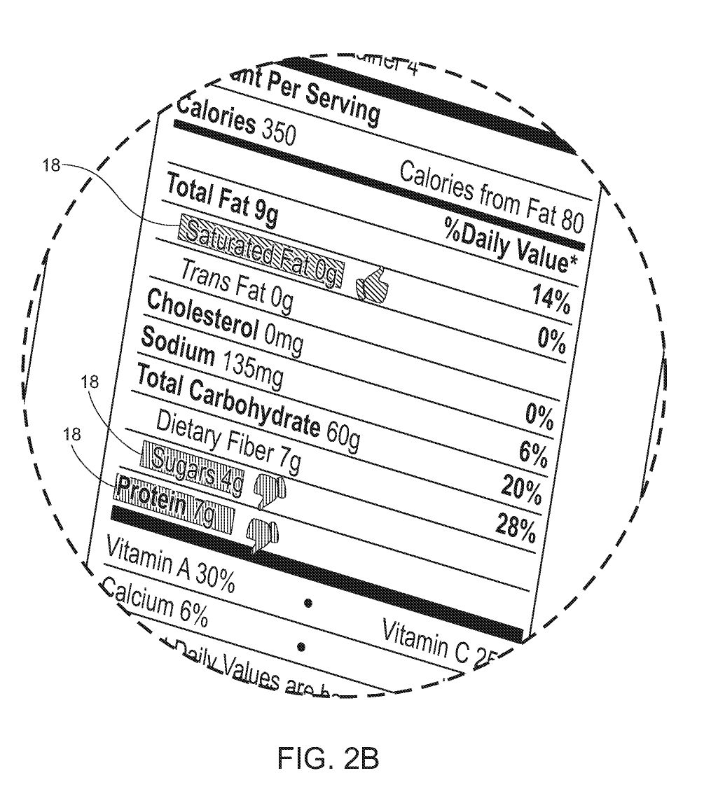

A result of the method is a recommendation that appears on the screen of the smartphone. FIG. 2A is a perspective view of a consumer 10 imaging a nutrition or food package label 12 on a package of food 14, whereby software loaded into device 16 (e.g. the smartphone) embodied by, for example, system 1 of FIG. 1 converts selected nutritional values listed on food package label 12 to recommendations 18 by use of a central processing unit (CPU) such as CPU 5, shown in FIG. 1, that links OCR 3, that, in combination with CPU 5, selects at least one nutritional value from food package label 14 (FIG. 2A) by optical character recognition that, by one embodiment of the method of the invention, converts data from database 7 (FIG. 1) to a recommendation that appears as an image shown on display screen 9 (FIG. 1), also shown as display screen 22 of FIG. 2A. Image, or images 18 of the recommendation, or recommendations, in one embodiment of the method of the invention are superimposed onto image 20 of food label 14 on screen 22 of device 16 simultaneously with display on screen 22 of a live image of the nutritional value as it appears on the package of food. In other words, if the optical character recognition component of the system ceases to recognize the selected nutritional value, such as by movement of the device, or smart device, housing the optical recognition device, away from the nutritional value or the package label, then the recommendation will cease, and the recommendation will no longer appear on the display of the device, thereby, at least in one embodiment, providing recommendations that are displayed on screen 22 of device 16 in real time (i.e., while consumer 10 is holding device 16 up to label 12 to capture image 20 of label 14). FIG. 2B is a detail of FIG. 2A, showing recommendations associated with selected nutritional values listed on food label 14. The recommendations can be, for example, binary, whereby, as shown in FIG. 2B, a positive recommendation is indicated by one color, such as green (indicated by one cross-hatching), and a negative recommendation can be indicated by another color, such as red (indicated by a different cross-hatching). Optionally, or alternatively, the positive recommendation can be indicated by an icon, such as a “thumbs-up,” which also can be color coded (e.g. green for favorable), and the negative recommendation can be indicated by a different, but related icon, such as a “thumbs-down,” which can also be color coded (e.g. red for unfavorable). The display of the recommendation can be superimposed on an image of the package label on a screen of device 16, by the method of the invention.

In one embodiment, the method of the invention for making a recommendation for a package of food to a consumer includes selecting at least one nutritional value from a food package label of a package of food by optical character recognition of a representation of the selected nutritional value as it is presented on the package of food. The recommendation can be, for example, whether to purchase a package of food bearing the food package label, or whether to include the food from the package bearing the package label in a dish or meal that is to be prepared. The nutritional value is selected from the food package label by, for example, holding a suitable imaging device, such as a handheld, or mobile, device, such as a so-called “smartphone,” that can be generally described as a mobile computing device. The mobile computing device selects at least one nutritional value from the food package by optical character recognition of a representation of the selected nutritional value as it is presented on the package of food. The at least one selected nutritional value is compared to at least one recommended nutritional value corresponding to the selected nutritional value to form at least one comparison value. A recommendation is generated from the at least one comparison value and is displayed as a recommendation to the consumer of the food package bearing the food package label. The recommendation appears simultaneously with the image of the food package label and appears with, or is superimposed over the image of the food package label on the display screen of the mobile computing device. In other words, if the optical character recognition component of the system ceases to recognize the selected nutritional value, such as by movement of the device, or smart device, housing the optical recognition device, away from the nutritional value or the package label, then the recommendation will cease, and the recommendation will no longer appear on the display of the device. The method thereby makes a recommendation of the food package to the consumer in real time, as augmented reality.

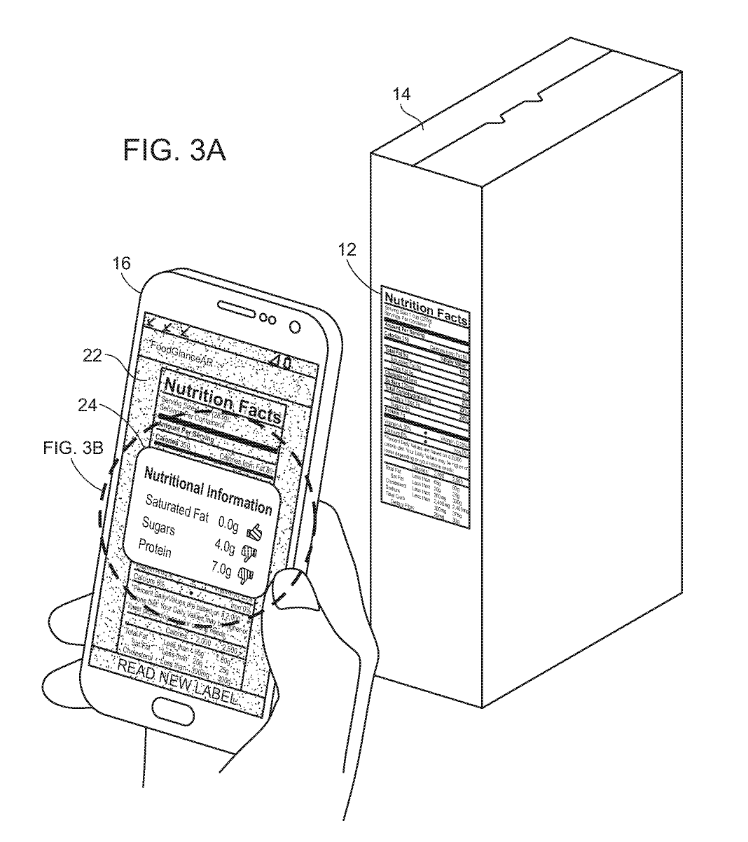

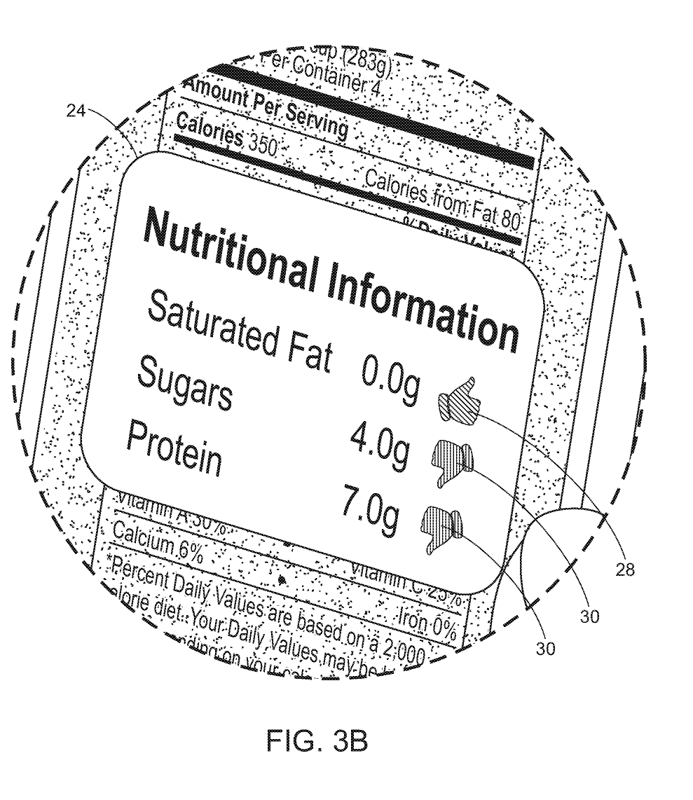

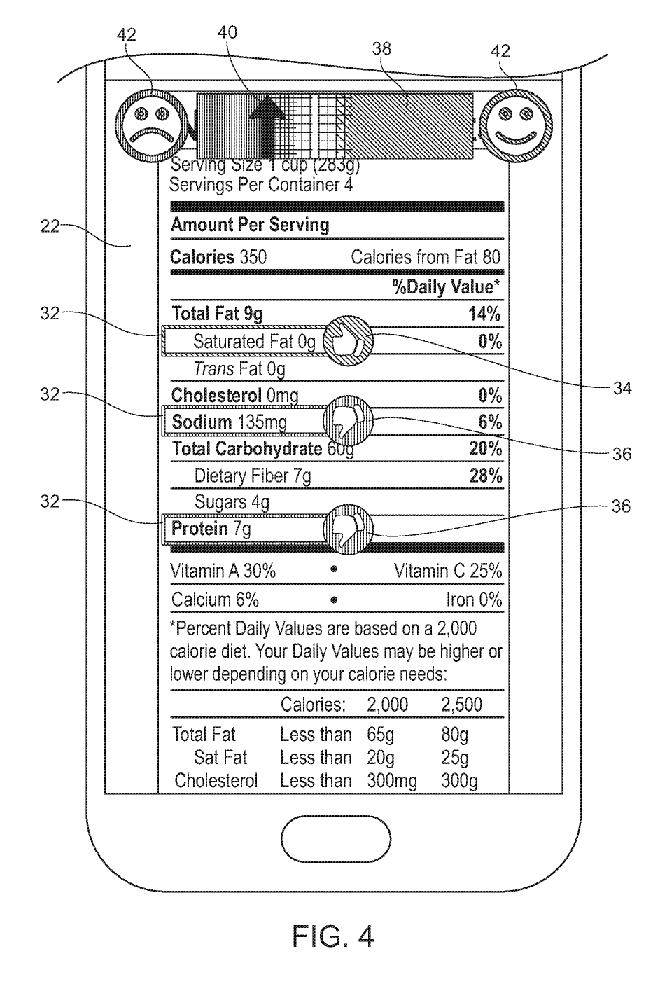

In one embodiment, the recommendation is displayed on screen 22 of device 16, as referenced above with respect to FIGS. 2A and 2B. In another embodiment, the method of the invention includes the step of displaying the recommendations as a separate listing 24, either apart from or superimposed upon an image of nutrition label 12. For example, as shown in FIGS. 3A and 3B recommendations are represented in real time as “thumbs-up” or “thumbs-down” icons, 28,30 respectively, which can be color coded, as indicated by the distinct cross-hatchings, made more clear in FIG. 3B. In another embodiment, shown in FIG. 4, the selected nutritional values are framed by images 32 superimposed over the values as they appear on the nutrition label 12. Image frames 32 can be color coded, as indicated by distinct cross-hatchings. Corresponding positive and negative icons 34,36 can also be color coded to match color coding of image frames 32.

In yet another embodiment, not shown, comparison values are simultaneously employed to generate an overall recommendation that is indicated as an upwardly-pointed arrow along a color spectrum flanked by “unhappy” and “happy” faces to thereby indicate to the consumer the strength of the recommendation.

Where there is only a single comparison value, the recommendation and the comparison value may be the same. Alternatively, where the consumer selects a plurality of nutritional values from the food package label, a plurality of comparison values will be generated, some or all of which will be displayed to the consumer, at least a portion of which will be incorporated into a recommendation that is also displayed to the consumer, such as on a screen of a mobile device. The individual nutritional comparison values can be, in one embodiment, weighted by the consumer, such as by virtue of relative importance to the consumer, either by, for example, dietary or other requirements, or by personal preference, or both, so that the recommendation reflects the relative weighting of the plurality of nutritional comparison values employed to generate the recommendation. In one embodiment, the comparison values and the recommendation are superimposed on a real time image of the food package label. In a specific embodiment, the comparison values are located proximate to the corresponding representations of the selected nutritional values as they appear on the label, while the overall recommendation is located on the display screen in a position that does not interfere with the image of the food package label itself.

In one embodiment, the at least one recommended nutritional value is derived from a pre-existing set of recommended nutritional values. Examples of sources of the pre-existing set of recommended nutritional values can be a set of recommended nutritional values selected at least in-part by the consumer from a pre-existing list that is maintained and updated by some third party. In another embodiment, the pre-existing set of nutritional values is solely prepared by a third party and maintained by that third party. In yet another embodiment, the list of selected nutrients is prepared solely and maintained solely by the consumer. Examples of said third parties include the federal government, such as the U.S. Department of Health and Human Services and the U.S. Department of Agriculture. Examples of suitable pre-existing sets of nutritional values maintained by the federal government include Dietary Guidelines for Americans, 8th Edition (DGA).

In one embodiment, the list of nutritional values includes values selected by the consumer from a pre-existing list of nutritional values made available by the federal government, such as the U.S. Department of Health and Human Services, and the U.S. Department of Agriculture. In this embodiment, the consumer is able to, at the discretion of a consumer, amend the list of nutritional values selected for optical character recognition from representations of those selected nutritional values as they are presented on the package of food.

In one embodiment, the at least one recommended nutritional value is at least partially dependent upon at least one physical feature of the consumer. For example, the recommended nutritional value to which the nutritional value selected from the food packages compared could depend upon at least one physical feature of the consumer that is specific to the consumer and that might influence the recommendation generated and displayed to the consumer, according to the consumer's choice of those physical features. In one embodiment, the physical feature is a combination of the members of the group of physical factors consisting of age, sex, weight, diabetic status, cholesterol status, allergen sensitivity, gluten sensitivity, and physical activity level. In still another embodiment, the consumer maintains a plurality of pre-existing sets of nutritional values, and the method further includes the step of selecting the pre-existing sets of nutritional values prior to generation of the recommendation. In one embodiment, the pre-existing sets of nutritional values is each at least partially defined by at least one member of the group consisting of food type, and food state. For example, in one embodiment, the food type can include at least one member of the group consisting of meat, seafood, baked goods, fruit, vegetables, pasta, condiments, cereal, coffee, and dairy products. In one embodiment, the food state is defined as the condition of the food and can include, for example, at least one member of the group consisting of fresh, frozen, canned, cured, and salted foods.

In yet another embodiment, the method further includes the step of identifying by optical character recognition at least one secondary value on the food package. In one embodiment, the secondary value is defined as an aspect of the packaged food item that does not necessarily connote the food type or condition of the food, such as, for example, at least one member of the group consisting of brand, product name, ingredients, expiration date, and source. In a specific embodiment, the source is defined as a physical or cultural origin of the packaged food, such as, for example, at least one member of the group consisting of local, country of origin, and ethnicity.

In still another embodiment, the method further includes the step of displaying the secondary value with the recommendation. In a specific embodiment, the method includes the step of combining the at least one secondary value with the at least one comparison value to generate the recommendation.

In one embodiment, the purchase recommendation is a binary choice, such as “purchase recommended,” or “purchase not recommended.” When the recommendation is for inclusion of a food for a labeled package of the food for a dish or a meal, the binary choice could be, for example, “include” or “don't include.” The binary choice can be presented in various forms, such as an icon, including, for example, a thumbs-up icon and a thumbs-down icon.

In another embodiment, the recommendation is displayed as a value on a scale, such as the position of an arrow or some other icon along a scale, such as a progression of primary colors.

The recommendation is displayed during optical recognition of the representation of the nutritional value on the food package label. The recommendation is displayed simultaneously with display of an image of the representation of the nutritional value. The recommendation is superimposed on the image of the representation of the nutritional value simultaneously with optical character recognition of the representation of the nutritional value that results in generation of the recommendation by the CPU, described above, thereby generating an augmented reality view. If the optical character recognition component of the system ceases to recognize the selected nutritional value, such as by movement of the device, or smart device, housing the optical recognition, device away from the nutritional value or the package label, then the recommendation will cease, and the recommendation will no longer appear on the display of the device.

In another embodiment, the recommendation is displayed in at least one of a color code and a shape that indicates a value associated with the recommendation. In a further embodiment, the recommendation is displayed as a color code that indicates the recommendation. In yet another embodiment, the recommendation is displayed as a shape that indicates the recommendation.

In one specific embodiment of the method, dietary guidelines are obtained from the United States Department of Health and Human Services and the United States Department of Agriculture web site or from the consumer. A recommended minimum intake amount of desired nutrients and maximum intake amount of undesired nutrients from the dietary guidelines chosen to be included in the database is stored in the device employed to conduct the method of the invention, which, optionally, can be updated, continuously, periodically, or upon request by the consumer receiving the recommendation by a suitable remote data base through an internet connection, or at some remote site. Nutrition facts on the nutrition label attached to the food product are scanned with the smartphone, or other imaging device, or microprocessor, or scanned using real time optical character recognition (OCR), employed by an optical character recognition module to extract nutrient values on the label in real time. A smartphone processor, for example, or the microprocessor, is employed to calculate percent daily values of nutrients by dividing and extracting values by recommended intake values, such as are obtained, for example, from a third party. The smartphone processor determines if an amount of each nutrient is labeled acceptable or nonacceptable to consumer using, for example, the FDA's “5-20 rule,” which is known and understood by those of skill in the art, whereby the nutrient is “acceptable” if the percent daily value of desired nutrient is more than 20% and the percent daily value of undesired nutrient is less than 5%, and that the food product is not recommended for purchase outside of these prescribed ranges. The real time location of the nutrient name and value is identified on the smartphone screen and, simultaneously, visual advice forwards nutrients as displayed in augmented reality directly over the nutrient locations or in a form of a pop-up display on the smartphone screen, highlighting the acceptable nutrients in green, and displaying a green thumbs-up next to their values and highlighting the unacceptable nutrients in red, and, optionally, or alternatively, displaying a red thumbs-down next to those values.

A flowchart for method steps of one embodiment of the method of the invention is presented in FIG. 4. As shown in FIG. 4, a database “A” 50 for threshold intake (“TIi”) is created, or derived, either from information listed, for example, by a third party, such as the United States Department of Health and Human Services and the United States Department of Agriculture 52 (collectively), from a suitable website, or as prescribed by the consumer to whom purchase recommendations are made by the method of the invention. Alternatively, the database “A” 50 is an assimilation of items derived from a combination of lists available from a third party and selections from that list, or separate lists generated by the consumer 54 (collectively).

An optical character recognition module 56 is employed, in one embodiment of the method of the invention, to generate an amount per serving of the nutrient and a nutrition table (APSi) 58 that is applied to the percent daily value of the nutrient. A percent daily value of a nutrient (PDVi) for each nutrient can be generated by comparing the APSi with the TIi (e.g., PDVi=(APSi/TIi*100)%) 60.

In this embodiment, decision 62 is made if the percent daily value for the desired nutrient is greater than 20% and optionally, additionally, or alternatively, if an undesired nutrient is less than 5%, then a positive purchase recommendation will be generated which will be displayed by the augmented reality module (“AR” module) or optical character recognition module (“OCR” module) 64 and displayed on the smartphone screen, optionally by highlighting that particular nutrient in green and placing a green thumbs-up icon next to its value on the display screen 66.

Alternatively, if the percent daily value for the desired nutrient is lower than 20% or, optionally, additionally, or alternatively, if an undesired nutrient is greater than 5%, of an undesired amount, then a negative recommendation would be generated 68 and displayed by the augmented reality module/OCR module on a smartphone screen that highlights the nutrient, over which the recommendation is superimposed, in red, and places a thumbs-down next to its value.

The benefits of the invention include, for example, real-time optical character recognition when scanning the label, wherein the OCR module automatically and continuously captures images of the label and extracts the text of interest to the consumer in making a purchase decision. The smartphone can remain in a camera mode, which then can be employed to superimpose the result over the product itself. In this way, it is combining as a seemingly simultaneous result the capture of an image and superimposition of a recommendation, making the method of this invention fast and accurate.

Further, the method of the invention has the benefit of identifying whether a product is acceptable to consume or not by, for example, applying the FDA's “5-20 rule.” The recommendation is based on the percent daily value that is obtained or calculated by nutrient values present in the database and dietary guidelines. The OCR module extracts all of the required data to process final results (e.g., advice/recommendation) and no additional template/product database is required to identify the product or values. Further, there is no need for use of a third-party database, thereby making the method of the invention usable without an internet connection.

Still another benefit is that augmented reality can, optionally superimpose visuals directly over the product by using a smartphone screen, for example, giving the sense to the user that it is a part of the product itself and that the consumer is directly interacting with the product in real time, also referred to as augmented reality. By making the product interactive, the method approves the usability and enhances the quality of the experience by the consumer.

In still another embodiment, a method of the invention is in the form of visual keys that highlight the nutrient and include, for example, a thumbs-up or thumbs-down recommendation to clearly indicate an acceptable or unacceptable value. The highlight and thumbs-up or thumbs-down can, optionally, also be color coded for ease of understanding.

Examples of suitable platforms for use of the method of the invention include operating systems, such as Android, iOS, Windows, etc. using a suitable software development kit (SDK) provided by the respective operating system developers (e.g., Google, Apple, Microsoft, etc.). The method of the invention employs a suitable optical character recognition module, such as any suitable module, plug-in or add-on that is compatible with the operating system of the software development kit to thereby provide a suitable optical character recognition technology (e.g., from Google Mobile Vision, Tesseract, etc.).

The augmented reality module is any suitable module, plug-in or add-on that is compatible with the selected operating systems software development kit to provide augmented reality technology (e.g., ARToolKit, ARKit, ARCore, etc.).

Dietary guidelines suitable for use include, for example, those published every five years for Americans by the United States Department of Health and Human Services and the United States Department of Agriculture.

The “FDA 5-20 rule,” is a general rule (or rule of thumb) employed by the Food and Drug Administration (FDA) to determine if a nutrient amount is acceptable or unacceptable based on a percent daily value.

Food manufacturers typically require placement of serving size on the labels if it is a manufactured food that is subject to nutritional labeling requirements. Serving size obtained from the FDA's reference amounts customarily consumed were in the RACC document and contains the procedure to determine the serving size for a food product. In one embodiment, the method of the invention does not require a factoring-in of a serving size calculation because the FDA's 5-20 rule is based on a percent daily value, which can determine if a food is acceptable to consume for any diet.

What is claimed is:

1. The method for making a recommendation to a consumer from a package of food, comprising the steps of:
a) selecting at least one nutritional value from a food package label of a package of food by optical character recognition of a representation of the selected nutritional value as it is presented on the package of food;
b) comparing the at least one selected nutritional value to at least one recommended nutritional value corresponding to the selected nutritional value to form at least one comparison value;
c) generating a recommendation from the at least one comparison value; and
d) displaying the recommendation for the food package to the consumer on a display simultaneously with the optical character recognition of the nutritional value, wherein the recommendation is displayed with, or is superimposed on an image on the display screen of the representation of the nutritional value, thereby making a recommendation of the food package to the consumer.

2. The method of claim 1, wherein the recommendation is whether to include the food from the package of food bearing the food package label in a preparation of food.

3. The method of claim 1, wherein the recommendation is whether to purchase the package of food bearing the food package label.

4. The method of claim 1, further including the step of displaying the at least one comparison value associated with the recommendation.

5. The method of claim 1, wherein a plurality of nutritional values are selected from the food package label, and a plurality of comparison values are formed corresponding to the respective selected nutritional values.

6. The method of claim 5, further including the step of combining at least a portion of the plurality of nutritional comparison values to generate the recommendation that is displayed.

7. The method of claim 6, further including the step of displaying the comparison values with the recommendation.

8. The method of claim 1, wherein the at least one recommended nutritional value is derived from a pre-existing set of recommended nutritional values.

9. The method of claim 8, wherein the pre-existing set of nutritional values is maintained by a third party.

10. The method of claim 8, wherein the pre-existing set of nutritional values is at least partially maintained by the consumer.

11. The method of claim 10, wherein the at least one recommended nutritional value is at least partially dependent upon at least one physical feature of the consumer.

12. The method of claim 11, wherein the at least one physical feature of the consumer is at least one member of the group consisting of age, sex, weight, diabetic status, cholesterol status, allergen sensitivity, and gluten sensitivity, and physical activity level.

13. The method of claim 12, wherein the physical factor is a combination of at least a portion of the members of the groups of physical factors.

14. The method of claim 8, wherein the consumer maintains a plurality of pre-existing sets of nutritional values, and further including the step of selecting the pre-existing sets of nutritional values prior to generation of the purchase recommendation.

15. The method of claim 14, wherein the pre-existing sets of nutritional values is each at least partially defined by at least one member of the group consisting of food type, and state.

16. The method of claim 15, wherein the food type includes at least one member of the group consisting of meat, seafood, baked goods, fruit, vegetables, pasta, condiments, cereal, coffee, and dairy products.

17. The method of claim 15, wherein the state includes at least one member of the group consisting of fresh, frozen, canned, cured, and salted.

18. The method of claim 1, further including the step of identifying by optical character recognition at least one secondary value on the food package.

19. The method of claim 18, wherein the secondary value includes at least one member of the group consisting of brand, product name, ingredients, expiration date, and source.

20. The method of claim 19, wherein the source includes at least one member of the group consisting of local, country of origin, and ethnicity.

21. The method of claim 16, further including the step of displaying the secondary value with the recommendation.

22. The method of claim 21, further including the step of combining the at least one secondary value with the at least one comparison value to generate the recommendation.

23. The method of claim 1, wherein the recommendation is a binary recommendation.

24. The method of claim 1, wherein the recommendation is a value on a scale.

25. The method of claim 1, wherein the recommendation is displayed in at least one of a color code and a shape that indicates a value associated with the recommendation.

26. The method of claim 25, wherein the recommendation is displayed as a color code that indicates the recommendation.

27. The method of claim 25, wherein the recommendation is displayed as a shape that indicates the recommendation.

28. A system for making a recommendation to a consumer from a package of food, comprising:
a) an optical character recognition device;
b) a database;
c) a central processing unit linking the optical character recognition device and the database; and
d) a display screen,
wherein,
i. the optical character recognition device can select at least one nutritional value from a food package label of a package of food by optical character recognition of a representation of the selected nutritional value as it is presented on the package of food,
ii. the central processing unit can compare the at least one selected nutritional value to at least one recommended nutritional value in the database corresponding to the selected nutritional value to form at least one comparison value,
iii. the central processing unit can generate a recommendation from the at least one comparison value, and
iv. the recommendation for the food package to the consumer can be displayed on the display screen simultaneously with the optical character recognition of the nutritional value, wherein the recommendation is displayed with, or is superimposed on an image on the display screen of the representation of the nutritional value, thereby making a recommendation of the food package to the consumer.
